(19) United States
(12) Patent Application Publication
(10) Pub. No.: US 2003/0050712 A1
(43) Pub. Date: Mar. 13, 2003

(54) Structure of knee joint stressing device

(76) Inventor: Hsin Fa Shen, Taipei (TW)

Correspondence Address: HSIN FA SHEN, P.O. BOX 26-757, TAIPEI, R.O.C. 106, TW

(21) Appl. No.: 09/865,080

(22) Filed: Sep. 13, 2001

(51) Int. Cl. A61F 2/64

(52) U.S. Cl. 623/45

(57) ABSTRACT

The present invention provides an improved structure of a knee joint stressing device, which comprises a main body and a turning shaft. The main body is screwed to the turning shaft through an inner thread. The root of the thread is used as a lubricant storing space so that lubricant can exist on the contact surface more easily. Moreover, the thread surface is used to increase the contact area, hence reducing abrasion between components, extending lifetime, saving unnecessary components, and lowering the manufacturing cost.

### FIELD OF THE INVENTION

The present invention relates to an improved structure of a knee joint stressing device and, more particularly, to an improved structure of a knee joint stressing device used as a knee joint of an artificial limb of a disabled person, so that the artificial limb will not easily loosen and sway when he walks. Moreover, the present invention can reduce abrasion of the parts thereof to extend the lifetime, and requires fewer components to lower the manufacturing cost.

### BACKGROUND OF THE INVENTION

Along with continual progress of medical technology, remedy effect keeps improving, and rehabilitation after remedy becomes more advantageous, letting more and more disabled persons be capable of relying on transplants or artificial limbs to achieve effects of remedial rehabilitation. Especially, artificial limbs are more common, and can let the disabled move about more conveniently, hence compensating their bodily deficiencies and reducing harms to their bodies and minds.

Generally speaking, a prior art safety knee joint (shown in FIGS. 1 and 2) comprises a knee device la, a buffer device 2a, a shank device 3a, and a stressing device 4a. One side in the knee device la and the shank device 3a are connected to two ends of the buffer device 2a, respectively. The other side in the knee device la and the shank device 3a are connected to two ends of the stressing device 4a, respectively. The knee joint accomplishes bending effect of knee through the buffer device 2a and locking effect through the stressing device 4a. The knee device 1a and the shank device 3a can thus generate arresting effect within a certain range of bending due to weight of the human body, hence preventing the knee joint from bending too much to cause unnecessary hazards.

FIG. 3 shows an exploded perspective view of a stressing device of a prior art safety knee joint. The stressing device 4a has a stressing sleeve 40a and a turning shaft 41a. The stressing sleeve 40a is disposed in a through hole 42a of the stressing device 4a. The turning shaft 41a is disposed in the stressing sleeve 40a to be fixedly stressed by the stressing sleeve 40a. However, after the knee joint is used for a period of time, the stressing sleeve 40a, the turning shaft 41a, and the through hole 42a will abrade one another, hence enlarging the gaps between them.

Therefore, the knee joint will start loosening, and the stressing sleeve 40a cannot fixedly stress the turning shaft 41a so that the stressing device 4a gradually loses its locking effect. Moreover, the turning shaft 41a will also sway. Because the turning shaft 41a is pivotally joined on the shank device 3a, the shank device 3a will thus loosen to let the disabled stand unstably and thus fall down. Therefore, bearings 43a are disposed at two sides of the turning shaft 41a to let the turning shaft 41a be steady and not sway easily. Although the bearings 43a can prevent the above hazards, it has the following drawback.

Although the bearings 43a can let the turning shaft 41a not sway easily, when the stressing sleeve 40a loses its locking effect gradually due to abrasion, the stressing sleeve 40a needs to be replaced; otherwise, there is doubt of safety. Because the expensive knee joint is already a large economic burden to the disabled, if the cost of the bearings 43a is added, the manufacturing cost will be higher, hence further increasing economic burden to the disabled.

The reason of the above drawback can be described below. The stressing sleeve 40a, the turning shaft 41a, and the through hole 42a make sliding contact through circular arc planes. When they are sleeved together, lubricant will be pushed toward the outer edge to let lubricant not easily exist in the gaps of the contact planes thereof. Therefore, there is not sufficient lubricant film for lubrication and protection, resulting in serious abrasion between components. Moreover, the stressing sleeve 40a will easily abrade to reduce its lifetime, letting the turning shaft sway. More serious, the stressing sleeve 40a will lose its locking effect to cause unnecessary hazards.

Accordingly, the above stressing device of the safety knee joint in the prior art has inconvenience and drawbacks in practical use. The present invention aims to resolve the problems.

### SUMMARY OF THE INVENTION

The primary object of the present invention is to provide an improved structure of a knee joint stressing device, wherein a main body of the stressing device is screwed to a turning shaft through an inner thread. The root portion of the inner thread is used as a lubricant storing space to let lubricant more easily exist on the contact surface. Moreover, the surface of the thread is utilized to increase the contact surface for reducing abrasion between components, extending the lifetime, saving unnecessary components, and lowering the manufacturing cost.

The various objects and advantages of the present invention will be more readily understood from the following detailed description when read in conjunction with the appended drawings, in which:

### BRIEF DESCRIPTION OF THE DRAWINGS:

FIG. 1 is a perspective view of a prior art safety knee joint;

FIG. 2 is a side view of a prior art safety knee joint;

FIG. 3 is an exploded perspective view of a stressing device of a prior art safety knee joint;

FIG. 4 is an exploded perspective view of the present invention when being installed on a knee joint;

FIG. 5 is a perspective view of the present invention;

FIG. 6 is a cross-sectional view of the present invention;

FIG. 6A is an enlarged view of the part A shown in FIG. 6;

FIG. 7 is an exploded perspective view according to another embodiment of the present invention; and

FIG. 8 is a side view of a turning shaft according to another embodiment of the present invention.

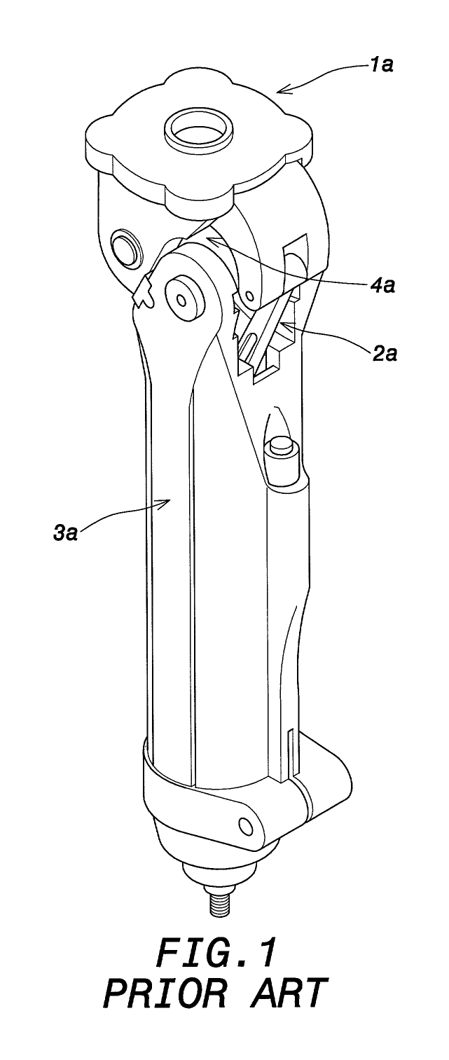

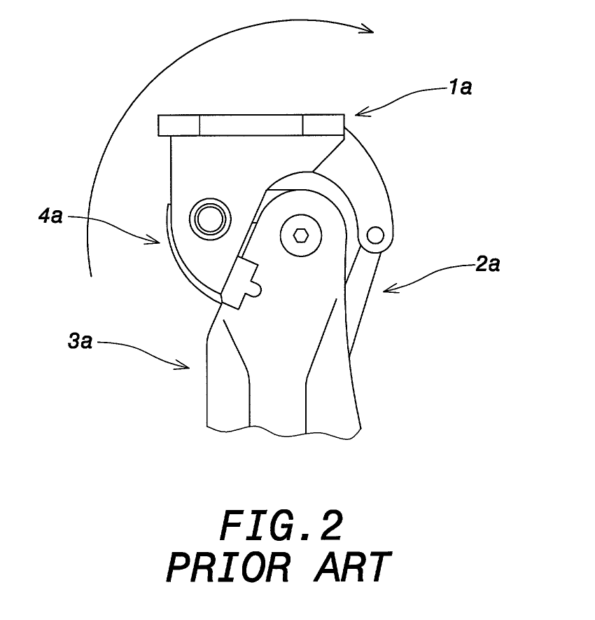

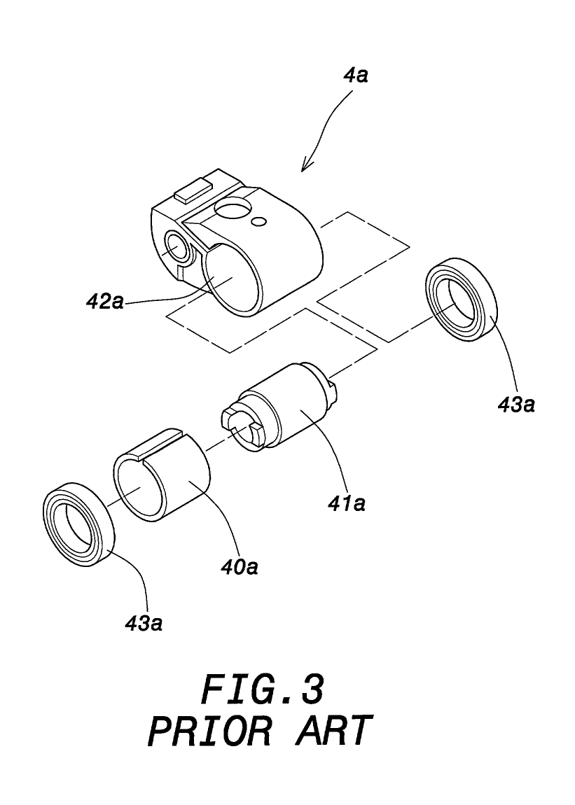

### DETAILED DESCRIPTION OF THE PREFERRED EMBODIMENTS

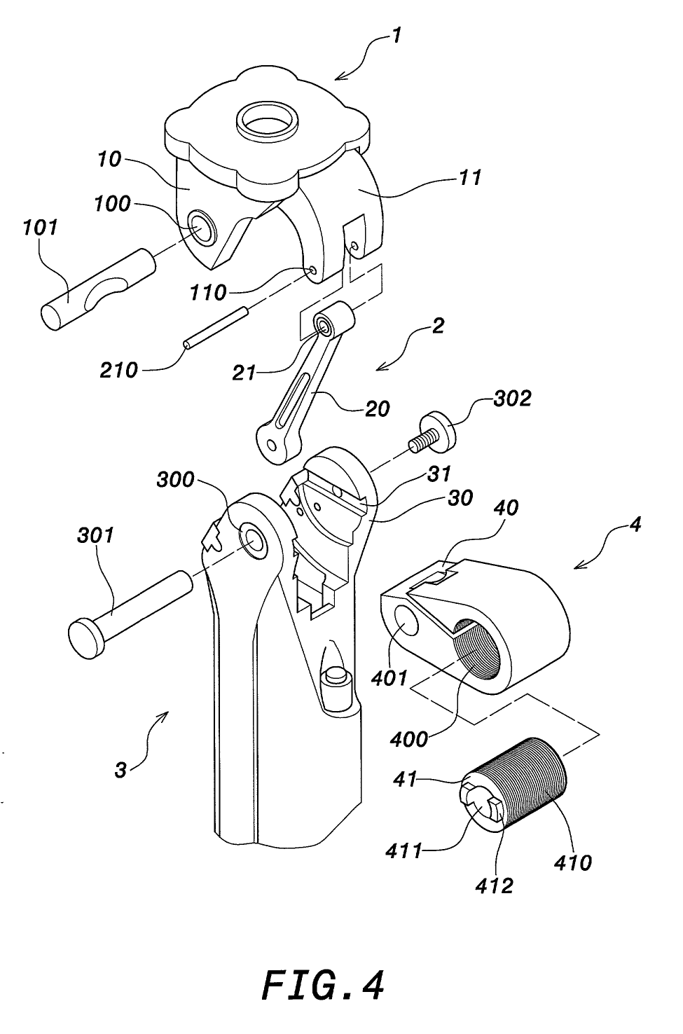

As shown in FIG. 4, the present invention provides an improved structure of a knee joint stressing device, which comprises a knee device 1, a buffer device 2, a shank device 3, and a stressing device 4. The top of the knee device 1 is used to join a thigh, and one side of the bottom thereof has a pivotal portion 10. The pivotal portion 10 has a pair of pivotal holes 100 disposed at opposite positions on two outside faces thereof. The other side of the bottom of the knee device 1 has a curved U-shaped connection block 11. The connection block 11 has a pair of through holes 110 disposed at opposite positions on two outside faces thereof.

The buffer device 2 comprises a buffer mechanism (not shown) and a knee axial rod 20. The buffer mechanism is connected at one side of the knee axial rod 20 through a restoring spring to jointly slide. Because the structure of the buffer mechanism is the same as a conventional one, it will not be further described. The top end of the knee axial rod 20 has a through hole 21 matched at an opening of the connection block 11 of the knee device 1 and corresponding to the through hole 110 of the connection block 11. A shaft 210 is used to pass through the through holes 21 and 110 to pivotally connect the knee device 1 and the buffer device 2 together.

The shank device 3 is a hollow bar with the buffer device 2 placed therein. The bottom of the shank device 3 is used to connect parts of artificial limbs such as ankles and soles. The top thereof has a recessed pivotal portion 30, which has a pair of axial holes 300 disposed at opposite positions on two raised outside faces thereof. A pair of slide grooves 31 are disposed at the positions of the axial holes 300 on two inside faces of the pivotal portion 30.

The stressing device 4 has an inner thread 400 disposed at the rear end of a main body 40 thereof. The inner thread 400 is matched with a turning shaft 41, which is screwed into the inner thread 400 of the stressing device 4 through an outer thread 410. The axis of the turning shaft 41 has an axially extending axial hole 411. The turning shaft 41 has slide blocks 412 on two end faces thereof to correspond to the slide grooves 31 of the shank device 3. The slide blocks 412 slide in the slide grooves 31, so that an axial rod 301 penetrates through the axial holes 300 of the pivotal portion 30 and the axial holes of the turning shaft 41, and a stud 301 is screwed to the axial rod 301, hence letting the shank device 3 and the stressing device 4 be joined together. The front end of the main body 40 of the stressing device 4 is connected with the knee device 1. The front end of the main body 40 has a pivotal hole 401 corresponding to the pivotal hole 100 of the pivotal portion 10. A pivotal rod 101 is used to pass through the pivotal holes 100 and 401 to pivotally connect the knee device 1 and the stressing device 4 together.

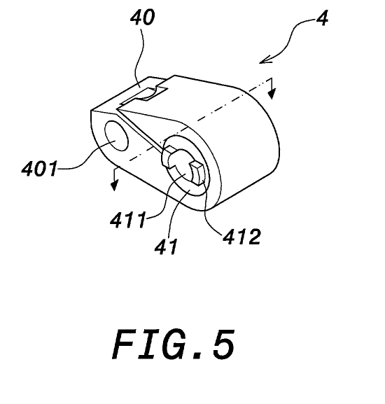

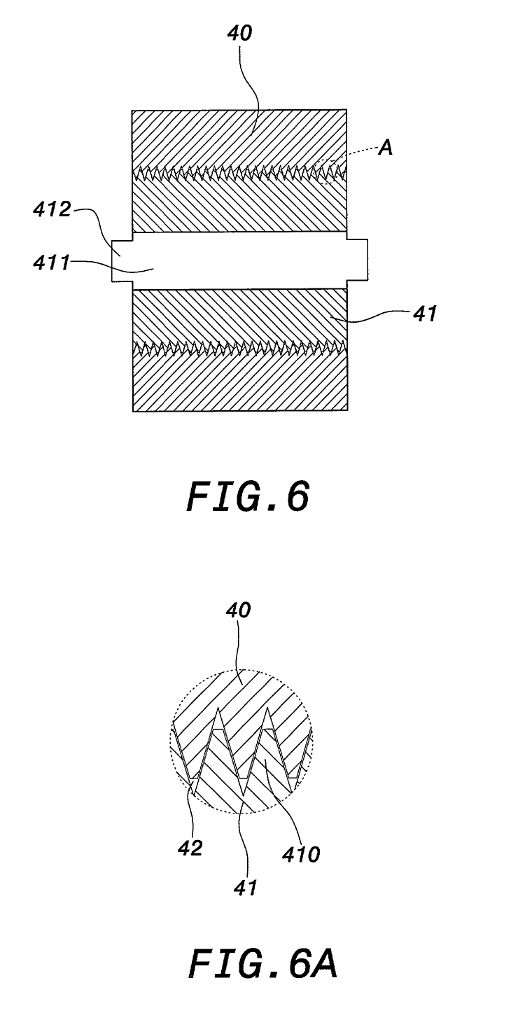

As shown in FIGS. 5 and 6, when the main body 40 of the stressing device 4 is screwed to the turning shaft 41, a larger gap 42 (shown in FIG. 6A) will be formed at the root of the thread 400 and the outer thread 410. The gap 42 can be used as a lubricant storing space for bringing lubricant to the thread surface to form lubricant film for lubrication and protection, hence reducing the surface abrasion and extending the lifetime.

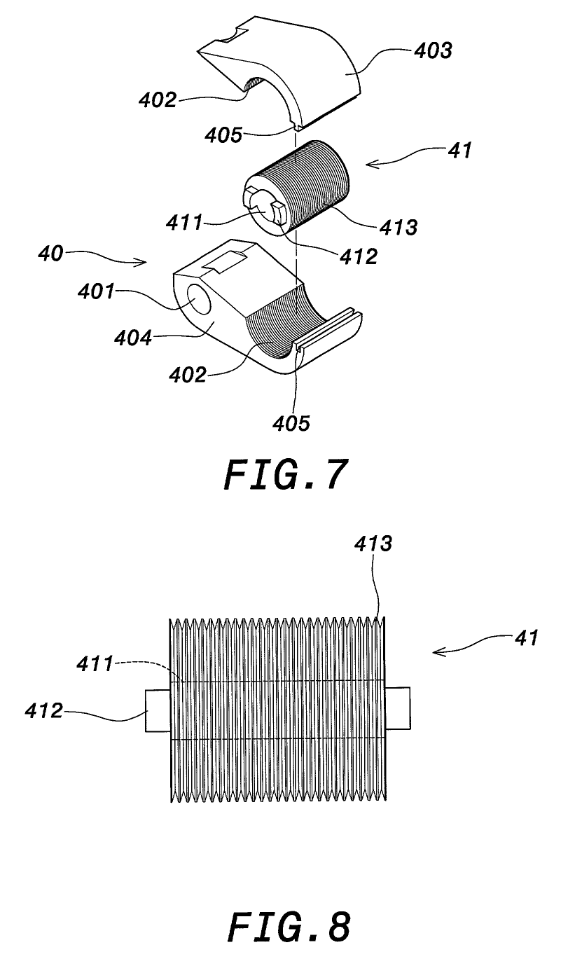

In another embodiment (shown in FIGS. 7 and 8) of the present invention, the outer wall of the turning shaft 41 has an annular body 413 having V-shaped grooves, and the rear end of the main body 40 has a corresponding annular hole 401 also having V-shaped grooves. Moreover, The main body 40 is divided into an upper main body 403 and a lower main body 404. A joining portion 405 is used to connect the upper main body 403 and the lower main body 404 to achieve the same effect as the above embodiment of the present invention.

To sum up, an improved structure of a knee joint stressing device of the present invention comprises only a main body and a turning shaft. The count of components thereof is smaller, thus saving the molding cost and lowering the manufacturing cost. Moreover, threads are used for connection to increase the area of the contact surface and thus reduce abrasion. Because the taper angle of the thread is small, more force-saving effect can be achieved as compared to a plane, thereby effectively enhancing the locking effect and safety. Additionally, a lubricant storing space is obtained so that there is sufficient lubricant film on the contact surfaces between components for lubrication and protection.

Although the present invention has been described with reference to the preferred embodiments thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

I claim:

1. An improved structure of a knee joint stressing device comprising a main body and a turning shaft, the rear end of said main body having an inner thread or an annular hole having grooves, the outer wall of said turning shaft having an outer thread or an annular body having grooves, said outer thread or said annular body being screwed to said inner thread or said annular hole of said main body, two end faces of said turning shaft having slide blocks corresponding to slide grooves of a shank device, said slide blocks sliding into said slide grooves to let said shank device and said stressing device be pivotally joined together, the front end of said main body being pivotally joined with a pivotal portion of a knee device.
